(19) United States
(12) Patent Application Publication
(10) Pub. No.: US 2023/0168190 A1
(43) Pub. Date: Jun. 1, 2023

(54) GAS SENSORS USING NON-DISPERSIVE INFRARED (NDIR) MATERIALS

(71) Applicant: Lumileds LLC, San Jose, CA (US)

(72) Inventors: Hisashi Masui, Newark, CA (US); Oleg Borisovich Shchekin, San Francisco, CA (US); Franklin Chiang, Campbell, CA (US); Emma Dohner, Menlo Park, CA (US)

(21) Appl. No.: 17/986,196

(22) Filed: Nov. 14, 2022

Related U.S. Application Data
(60) Provisional application No. 63/284,999, filed on Dec. 1, 2021.

(51) Int. Cl. G01N 21/3504; G01N 33/00

(52) U.S. Cl. CPC G01N 21/3504; G01N 33/0027; G01N 2201/062

(57) ABSTRACT

In a gas sensing system, an emitter emits light through a gas toward a concave reflective surface. The reflective surface reflects the light toward a sensor while light that passes through a porous scattering material is scattered. The surface of the reflective surface provides a diffusion of the light. A concentration of the gas is detected by the sensor. The scattering material may be permeable or non-permeable to the gas. The scattering and reflecting of the light increases the distance the light travels from the emitter to the sensor to increase absorption of the light by the gas.

### PRIORITY CLAIM

This application claims the benefit of priority to U.S. Provisional Patent Application Ser. No. 63/284,999, filed Dec. 1, 2021, which is incorporated herein by reference in its entirety.

### TECHNICAL FIELD

The present disclosure relates to detecting a concentration of a gas.

### BACKGROUND

There is ongoing effort to improve detecting a concentration of a gas. In particular, gas detectors in many commercial applications may be bulky and difficult to install.

### BRIEF DESCRIPTION OF THE DRAWINGS

FIG. 1 shows a schematic drawing of an example of a gas sensing system, in accordance with some embodiments.

FIG. 2 shows a cross-sectional side view of an example of a gas sensing system, in accordance with some embodiments.

FIG. 3 shows a cross-sectional side view of an example of a gas sensing system, in accordance with some embodiments.

FIG. 4 shows a cross-sectional side view of an example of a gas sensing system, in accordance with some embodiments.

FIG. 5 shows a cross-sectional side view of an example of a gas sensing system, in accordance with some embodiments.

FIG. 6 shows a cross-sectional side view of an example of a gas sensing system, in accordance with some embodiments.

FIG. 7 shows a cross-sectional side view of an example of a gas sensing system, in accordance with some embodiments.

FIG. 8 shows a cross-sectional side view of an example of a gas sensing system, in accordance with some embodiments.

FIG. 9 shows a cross-sectional side view of an example of a gas sensing system, in accordance with some embodiments.

FIG. 10 shows a cross-sectional side view of an example of a gas sensing system, in accordance with some embodiments.

FIG. 11 shows a cross-sectional side view of an example of a gas sensing system, in accordance with some embodiments.

FIG. 12A shows a cross-sectional side view of a gas sensing system, in accordance with some embodiments.

FIG. 12B shows a cross-sectional side view of another gas sensing system, in accordance with some embodiments.

FIG. 12C shows a cross-sectional side view of another gas sensing system, in accordance with some embodiments.

FIG. 13 shows a cross-sectional side view of an example of a gas sensing system, in accordance with some embodiments.

FIG. 14 shows a cross-sectional side view of an example of a gas sensing system, in accordance with some embodiments.

FIG. 15 shows a flowchart of an example of a method for measuring a concentration of a gas in a gas sample, in accordance with some embodiments.

FIG. 16A shows a cross-sectional side view of a gas sensing system, in accordance with some embodiments.

FIG. 16B shows a cross-sectional side view of a gas sensing system, in accordance with some embodiments.

FIG. 16C shows a cross-sectional side view of another gas sensing system, in accordance with some embodiments.

FIG. 16D shows a cross-sectional side view of a gas sensing system, in accordance with some embodiments.

FIG. 16E shows a cross-sectional side view of a gas sensing system, in accordance with some embodiments.

Corresponding reference characters generally indicate corresponding parts throughout the several views. Elements in the drawings are not necessarily drawn to scale. The configurations shown in the drawings are merely examples and should not be construed as limiting the scope of the disclosed subject matter in any manner.

### DETAILED DESCRIPTION

Gas detection is becoming more common for a variety of applications. For example, detecting concentration levels of methane can help guide downstream decisions in the fields of industrial oil and gas exploration, safety, climate change, and others. Detecting concentration levels of formaldehyde and/or volatile organic compounds (VOCs) can help guide downstream decisions in the fields of air quality, safety, and others. Detecting concentration levels of carbon dioxide can help guide downstream decisions in the fields of smart buildings, air quality, capnography, climate change, and others. Detecting concentration levels of carbon monoxide and/or nitrogen dioxide can help guide downstream decisions in the fields of safety and others. Detecting concentration levels of ammonia, sulfur hexafluoride, and/or volatile organic compounds can help guide downstream decisions in the fields of refrigeration, electrical systems, and others. Detecting concentration levels of glucose can help guide downstream decisions in the fields of medicine and others.

Some gas detection systems can make use of a natural absorption of a gas material. For example, methane is found to be absorbent at a wavelength of about 3.3 microns. When a gas sample is illuminated with light at the wavelength of about 3.3 microns, methane in the gas sample may absorb some of the illumination. A sensor or detector in the gas detection system may measure the illumination remaining after the illumination passes through the gas sample.

One category of these illumination/detection gas detection systems can be based on the Beer-Lambert Law. In this category, the gas detection system illuminates the gas with light at or near the absorbent wavelength (or with light having a spectrum that includes the absorbent wavelength) and detects how much of the illuminating light passes through the gas sample. Based on the fraction of illuminating light that emerges from the gas sample, the gas detection system can calculate a concentration level of the particular gas in the gas sample.

For gas detection systems based on the Beer-Lambert Law, the sensitivity and/or accuracy of the system can scale with an optical path length over which the absorption can occur. As a result, gas detection systems with larger gas chambers tend to be more sensitive and/or more accurate than those with smaller gas chambers. For example, in a gas detection system in which the gas chamber is hollow, the illumination can progress in a straight line through the gas chamber, such that the optical path length can be comparable to a dimension of the gas chamber. However, it is desirable to shrink the size of gas detection systems to enable incorporation into more consumer goods, such as in appliances including heating, ventilation, and air conditioning (HVAC) systems or refrigeration systems, electronics such as smart speakers, or automobile systems (such as in a fuel system, an in-cabin ventilation system, and/or an exhaust system), and others. However, shrinking the gas detection systems to fit into smaller spaces can compromise the sensitivity and/or accuracy of the system.

To improve the sensitivity and/or accuracy of the system, various embodiments of the gas detection systems described herein dispose a porous solid scattering material in the gas chamber and/or in a wall surrounding the gas chamber, so that a gas sample can permeate hollow spaces within the porous scattering material. In various embodiments, the scattering material can be transparent at about the illuminating wavelength (e.g., the wavelength at which the gas material is absorbent).

The scattering material can greatly increase an optical path length of illuminating light that passes through the scattering material, compared with a single pass through a given volume (e.g., a linear dimension of the porous scattering material, or a path that would be taken if the space occupied by the porous scattering material were instead hollow). For example, in various embodiments of the gas detection system described herein, the scattering material can redirect the illumination multiple times within the scattering material. The actual optical path traversed by the illumination in the scattering material can be orders of magnitude larger than the actual size of the scattering material. As a result, the optical path length experienced by the illumination can be significantly greater than a dimension of the gas chamber, such by a factor of 10, 100, or more.

As a result of locating the porous scattering material inside the gas chamber and/or in a wall of the gas chamber, the gas chamber size can be decreased significantly, while still maintaining a sensitivity/accuracy comparable to what would be achieved by a system having a hollow gas chamber. Consequently, the gas detection systems described herein, which can dispose a porous scattering material in the gas chamber and/or in a wall of the gas chamber, can be significantly smaller than comparable systems that have a hollow gas chamber (optionally with gas impermeable walls), while achieving sensitivity/accuracy performance comparable to systems that have a hollow gas chamber (optionally with impermeable walls).

A gas sensing system can allow a gas sample to permeate hollow spaces within a porous scattering material. The porous scattering material can be substantially transparent at an illumination wavelength. An emitter can illuminate the porous scattering material and the gas sample with light having a spectrum that includes the illumination wavelength. A sensor can detect a level of light that has traversed the porous scattering material. Using an application of the Beer-Lambert Law, the system can determine a concentration of the gas material in the gas sample. The scattering can greatly increase an optical path length through the porous scattering material, compared with a linear dimension of the porous scattering material (e.g., a path that would be taken if the space occupied by the porous scattering material were instead hollow). The increased optical path length can allow a gas chamber to shrink in size, thereby decreasing a size of the gas sensing system without a corresponding decrease in a sensitivity and/or an accuracy of the system.

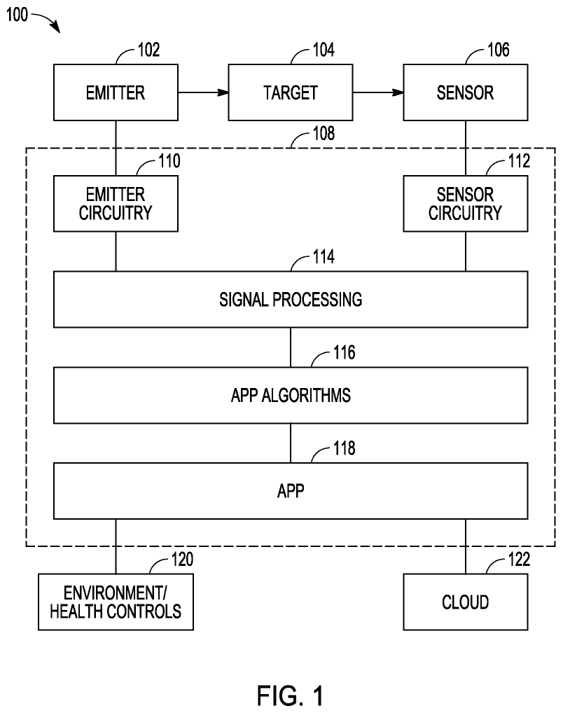

FIG. 1 shows a schematic drawing of an example of a gas sensing system 100, in accordance with some embodiments. As shown in the example of FIG. 1, an emitter 102 can emit light toward a target 104 (which may also be referred to herein as a target volume). The target 104 can include a porous scattering material, such as disposed in a gas chamber and/or in one or more walls of a gas chamber, so that a gas sample can permeate hollow spaces within the porous scattering material, such as inside the gas chamber. A sensor 106 can detect light, emitted from the emitter 102, that has traversed through the target 104. At least one processor 108, coupled to the sensor 106, can determine a concentration of a specified gas material in the gas sample.

The emitter 102 can be selected to emit light that can include a wavelength that is relatively strongly absorbed by the gas material that is to be detected.

For example, methane has an absorption peak (e.g., a wavelength at which absorption is relatively large, compared to the absorption at adjacent wavelengths) at a wavelength of about 3.3 microns. To detect a concentration of methane in the gas sample, the emitter 102 can emit light at about 3.3 microns. Similarly, the emitter 102 can emit light at about 3.6 microns to detect formaldehyde and/or volatile organic compounds. The emitter 102 can emit light at about 4.3 microns to detect carbon dioxide. The emitter 102 can emit light at about 4.5 microns to detect carbon monoxide. The emitter 102 can emit light at about 4.7 microns to detect nitrogen dioxide. The emitter 102 can emit light at about 9 microns to detect ammonia, sulfur hexafluoride, and/or certain volatile organic compounds. The emitter 102 can emit light at about 10.4 microns to detect glucose. These numerical examples are provided as examples only. Other suitable wavelengths can also be used to detect other gas materials or compounds.

The emitter 102 can emit light having a spectrum that is relatively sharply peaked compared to a broadband emitter like an incandescent lamp, which can be affected/absorbed by many substances. A sharp emitter is useful, even if methane and formaldehyde absorptions are close, in cases where it is known that only one of the two gases exists. The emitter 102 can emit light having a spectrum that is relatively broad. The emitter 102 can emit light having a spectrum that includes the wavelength at which absorption of the gas material or compound is relatively high. The emitter 102 can emit light in the infrared portion, the visible portion, and/or the ultraviolet portion of the electromagnetic spectrum. The emitter 102 can emit light in the Middle Wavelength Infrared (MWIR) portion of the electromagnetic spectrum, with a wavelength range extending from about 3 microns to about 5 microns. The emitter 102 can emit light in the Long Wavelength Infrared (LWIR) portion of the electromagnetic spectrum, with a wavelength range extending from about 8 microns to about 14 microns.

In various embodiments, the emitter 102 can include one or more light-emitting diodes (LEDs). The one or more light-emitting diodes can include III-V semiconductor materials (or other semiconductor materials from, for example, II-VI columns). The one or more light-emitting diodes can include gallium antimonide (GaSb), indium phosphide (InP), indium arsenide (InAs), or other suitable materials. The emitter 102 can include one or more lasers. The emitter 102 can include one or more broadband sources that are spectrally filtered.

The target 104 can include a porous scattering material, such as porous alumina, porous silicon, porous YAG, porous TiO₂, and others. The porous scattering material can be disposed in a gas chamber and/or in a wall or walls of the gas chamber, so that a gas sample can permeate hollow spaces within the porous scattering material inside the gas chamber and/or in the wall or walls of the gas chamber. The porous scattering material can be transparent, or substantially transparent, at the wavelength of the light emitted by the emitter 102. The porous scattering material can be transparent, or substantially transparent, at the wavelength at which the gas material is relatively absorbent. The target 104 and the porous scattering material can be sized and shaped in any suitable manner, as provided below.

The sensor 106 can detect light, emitted from the emitter 102, that has traversed through the target 104. The sensor 106 can include one or more pixels (e.g., detector elements or sensor elements) or other types of sensors known in the art. In some embodiments, the sensor 106 can be separate from the emitter 102. The sensor 106 can include one or more sensor elements that are formed from a same or similar semiconductor material (e.g., III-V compound semiconductors) that is used in the emitter 102.

The sensor 106 can optionally be formed integrally with the emitter 102. For example, the sensor 106 and the emitter 102 can both be formed as light-emitting diodes in a single array or in a single integral package. The emitter 102 can be forward biased. The sensor 106 can be reverse biased. Other configurations can also be used.

The processor 108, coupled to the sensor 106, can determine a concentration of a specified gas material in the gas sample. The processor 108 can include emitter circuitry 110 that can drive the emitter 102. The processor 108 can include sensor circuitry 112 that can determine a power level of light that strikes the sensor 106. The sensor circuitry 112 can optionally include, for example, an analog-to-digital converter (ADC), among others. The processor 108 can include signal processing circuitry 114 that can analyze an output of the sensor circuitry 112. For example, the signal processing circuitry 114 can receive a value that represents a sensed optical power value, and can calculate, from the received value, a concentration level of the gas material in the gas sample. The signal processing circuitry 114 can employ the Beer-Lambert Law to perform the calculation, although other suitable calculations can be performed; for a three-dimensional chamber a dedicated calibration or calculation is used to deduce adequate concentration values to be output. The processor 108 can include one or more application algorithms 116 that can serve as an interface between the signal processing circuitry 114 and an application that includes a user interface. The processor 108 can include one or more applications 118 that can interface with the one or more application algorithms 116. The one or more application algorithms 116 can communicate with one or more servers dedicated to the environment and/or health controls 120. The one or more application algorithms 116 can communicate with one or more servers connected to the cloud 122.

The gas sensing system 100 can optionally detect two or more gas materials in a same gas sample. The two or more gas materials can have different wavelengths at which the respective gas materials are relatively absorbent. The emitter 102 can emit light at respective two or more wavelengths. The sensor 106 can sense light at the two or more wavelengths. To sense at the wavelengths, the gas sensing system 100 can include one or more wavelength-sensitive filters, such as to direct one wavelength onto one sensor element and direct another wavelength onto another sensor element.

In some examples, the emitter 102 can optionally emit reference light having a spectrum that includes a reference wavelength different from the detection wavelength. In some embodiments, multiple reference beams of different wavelengths and/or detection beams of different wavelengths may be used for more precise measurements. A single wideband emitter may be used, or separate emitters may be used that are tuned to each desired wavelength. The gas sample can interact with the light at the detection wavelength but may not interact with the reference light at the reference wavelength. The sensor 106 can optionally detect at least some of the reference light. The at least one processor 108 can use the level of the reference light at the sensor 106, in addition to the level of the detection light at the sensor 106, to determine the concentration of the gas material in the gas sample. In some examples, for which the gas sensing system 100 can sense two different gas materials, the emitter can emit a first wavelength and a second wavelength. The wavelengths can be selected such that a first gas interacts with the first wavelength but not the second wavelength and a second gas interacts with the second wavelength but not the first wavelength. Light at the second wavelength can serve as a reference for detecting the first gas, while light at the first wavelength can serve as a reference for detecting the second gas. Other combinations can also be used. If multiple reference beams and/or detection beams are used for each gas as above, the measurements may be cross-correlated to provide enhanced precision.

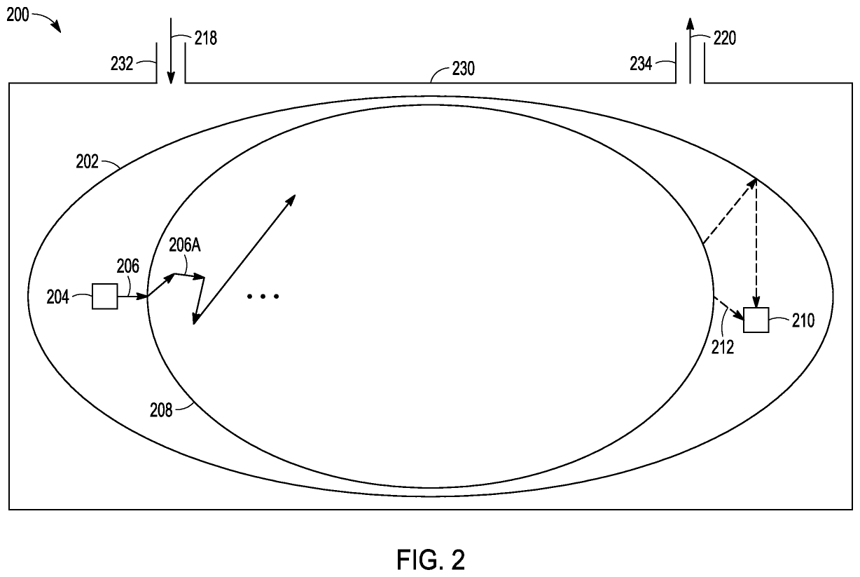

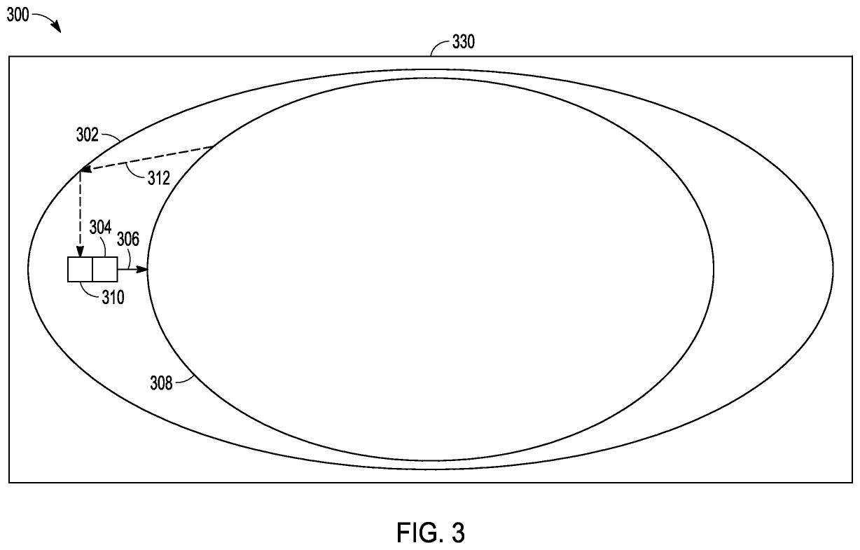

FIG. 2 shows a cross-sectional side view of an example of a gas sensing system 200, in accordance with some embodiments. FIG. 2, as well as FIGS. 3-8 below, omits the circuitry to operate electronics in the gas sensing system 200; any suitable circuitry can also be used as will be recognized by a person of ordinary skill in the art upon reading and understanding the disclosed subject matter.

The gas sensing system 200 contains a gas chamber 230 with a reflective surface 202, as well as a light emitter 204, light sensor 210, and a target 208. The reflective surface 202 may be substantially reflective at all wavelengths of interest. FIG. 2 illustrates plumbing of the gas chamber 230, which is omitted in FIGS. 3-8 below. The plumbing can controllably pump a gas sample into the gas chamber 230 before a measurement has been taken and can controllably pump the gas sample out of the gas chamber 230 after the measurement has been taken. The gas sensing system 200 can include an intake 218 through an intake port 232 of the gas chamber 230. The gas sensing system 200 can include an outlet 220 through an outlet port 234 of the gas chamber 230.

The target 208 can include a porous scattering material disposed in therein, so that a gas sample can permeate hollow spaces within the porous scattering material of the target 208. As shown, the light 206a that enters the target 208 may scatter multiple times within the target 208 due to the porous scattering material, thereby increasing the path length of the light 206a by a substantial amount and increasing the absorption of the light 206a by the gas sample. In some embodiments, the plumbing may move the gas sample into and/or out of the target 208. The target 208 can be located between the light emitter 204 and the light sensor 210. In the example of FIG. 2, the target 208 can match the geometry of the reflective surface 202; other geometries, however, may be used, e.g., where the target 208 matches the geometry of the gas chamber 230. In some embodiments, the target 208 may not be gas permeable and may instead only scatter the light 206a. In either case, use of a target 208 that provides scattering of the light 206a may be able to reduce the size of the cavity of the gas chamber 230, e.g., from about 3-5 cm along a characteristic dimensions (e.g., long axis) to less than about 1 cm while still providing similar or enhanced sensitivities (e.g., being able to determine CO₂ concentrations between 400 ppm and 420 ppm).

The reflective surface 202 may be concave, as shown in FIG. 2. The reflective surface 202 may, for example, be shaped to include all or part of an ellipsoid. Specifically, the ellipsoid has an elongation axis, such that a cross-section taken orthogonal to the elongation axis is generally circular, and a cross-section taken in a plane that includes the elongation axis is elliptical. The ellipsoid includes two foci that are spaced apart along the elongation axis. The reflective surface may, in some embodiments, be shaped such that every light ray, or nearly every light ray, that originates at one focus specularly reflects off the ellipsoidal reflective surface to be directed to the other focus. Further, the reflective surface may be shaped such that each undisturbed optical path between the two foci, including one reflection from the ellipsoidal reflective surface, has a same or nearly a same value of optical path length.

In some examples, the reflective surface 202 is a wall of the gas chamber 230. In other examples, such as the embodiment shown in FIG. 2, the reflective surface 202 can be contained inside the gas chamber 230, such as by being a structure that can reflect light without constraining the gas sample or defining a volume of the gas chamber 230. This may also allow the gas chamber 230 to have a shape that is conducive to being incorporated into other devices. In other examples, the reflective surface 202 can be outside the gas chamber 230, such as by forming the wall of the gas chamber 230 by a material that is transparent or substantially transparent at the emitted wavelength or wavelengths, such as plastic or glass, which constrains the gas sample but allows light to exit and re-enter the gas chamber 230.

In examples in which the reflective surface 202 is a complete or partial ellipsoid, a light emitter 204 can be located at or near a first focus of the ellipsoid. The light emitter 204 can be the same or similar to the emitter 102 of FIG. 1. The light emitter 204 may emit light generally in a direction toward the light sensor 210, in other embodiments the light may be emitted in substantially all directions simultaneously. The light emitter 204 can emit the light 206 into the target 208 and thus the gas sample. The light 206 can propagate away from the light emitter 204, through the gas sample, in a range of propagation directions. In embodiments in which the reflective surface 202 is smooth, the light 206 that strikes the reflective surface 202 may be specularly reflected (e.g., reflected without scattering, such that light with a single incident direction can reflect with a single exiting direction) from the reflective surface 202. The reflected light can also propagate through the target 208 and gas sample toward the second focus of the ellipsoid.

The reflective surface 202 may be smooth or may be textured. In one example, some or all of the reflective surface 202 may have a diffusing portion. The diffusing portion of the surface can diffusely reflect the light ray into multiple light rays having multiple propagation directions. To accomplish the diffuse reflection, the diffusing portion can be roughened, such as roughened at the scale of middle wavelength infrared (MVVIR) wavelengths, which would generate a randomized reflection from such a roughened surface for light in the range of MVVIR wavelengths. In some examples, the diffusing portion can be formed by disposing a reflecting layer, such as aluminum, on frosted glass. Other manufacturing techniques can also be used. Thus, the reflective surface 202 may provide specular reflection or, if roughened, non-specular reflection. In some embodiments, the reflective surface 202 may be formed, for example, from shaped stamped steel, alumina, or metalized plastics.

In the configuration of FIG. 2, the light sensor 210 can be located at or near the second focus of the ellipsoid to collect most or all of the light 212 emerging from the target 208 or reflected by the reflective surface 202. The light sensor 210 may the same as or similar to the sensor 106 of FIG. 1. The collected light will have traversed one of a range of optical paths from the first focus, to the reflective surface 202, to the second focus. Because the reflective surface 202 can be a complete or partial ellipsoid, the range of optical paths can have a path length that is the same or nearly the same, for most or all of the optical paths, regardless of propagation direction away from the light emitter 204. The light sensor 210 may, in some embodiments, be a relatively wideband detector that is able to detect light between 3-10 μm. In other embodiments, the light sensor 210 may be a detector that is more narrowly tailored to the wavelength range of light emitted by the light emitter 204.

In the configuration of FIG. 2, the light emitter 204 is oriented to emit light generally toward the light sensor 210 (e.g., with an angular emission pattern that peaks along an elongation axis of the ellipsoid and decreases at angles away from the elongation axis). It is possible to use different orientations for the light emitter 204, which can illuminate different portions of the reflective surface 202, and in turn illuminate different portions of the volume of the gas chamber. In some examples, it is possible to eliminate portions of the reflective surface 202 that would receive little or no illumination, which can reduce the size of the gas sensing system 200.

In the configurations of FIG. 2, the light emitter 204 and the light sensor 210 are located at or near different foci of the ellipsoid. Alternatively, both the light emitter 204 and the light sensor 210 may be at or near just one of the foci (similar to that shown in FIG. 3). In these examples, the collected light may have traversed one of a range of optical paths from the first focus, to the reflective surface 202, to and through the second focus, to the reflective surface 202, to return to the first focus. Because the reflective surface 202 can be a complete or partial ellipsoid, the range of optical paths can have a path length that is the same or nearly the same, for most or all of the optical paths, regardless of propagation direction away from the light emitter. The configuration with the light emitter 204 and the light sensor 210 both located at the same focus can have an optical path length that is about twice as large for the configuration in with the light emitter 204 and the light sensor 210 being at different foci.

The configuration of FIG. 2 uses a reflective surface 202 that is ellipsoidal or substantially ellipsoidal. As an alternative, the gas sensing system 200 can include a reflective surface that is shaped to include a complete or partial paraboloid. Specifically, the paraboloid has a central axis, such that a cross-section taken orthogonal to the central axis is generally circular, and a cross-section taken in a plane that includes the central axis is a parabola. The paraboloid includes one focus that is located along the central axis. The reflective surface may be shaped such that every light ray or nearly every light ray that originates at the focus specularly reflects off the paraboloid reflective surface to be directed to be parallel or substantially parallel to the central axis. Further, such a reflective surface may be shaped such that each optical path from the focus, including one reflection from the ellipsoidal reflective surface, to a plane that is orthogonal to the central axis, has a same or nearly a same value of optical path length.

FIG. 3 shows a cross-sectional side view of an example of a gas sensing system 300, in accordance with some embodiments. As above, FIG. 3 omits the circuitry to operate electronics in the gas sensing system 300 in addition to the plumbing. The gas sensing system 300 contains a gas chamber 330 with a (smooth or textured) reflective surface 302, as well as a light emitter 304, light sensor 310, and a target 308. The target 308 can include a porous scattering material disposed in therein, so that a gas sample can permeate hollow spaces within the porous scattering material of the target 308. As above, the light that enters the target 308 may scatter multiple times within the target 308 due to the porous scattering material. In this case, rather than the target 308 being located between the light emitter 304 and the light sensor 310 as in FIG. 2, the light emitter 304 and the light sensor 310 in FIG. 3 may be disposed adjacent to each other (e.g., back-to-back) at or near one of the foci of the ellipsoid. The light emitter 304 may emit light 306 generally in a direction toward the target 308, which propagates away from the light emitter 304, through the gas sample, in a range of propagation directions until the light 312 exits the target 308 and reaches the light sensor 310.

In the example of FIG. 3, the target 308 can match the geometry of the reflective surface 302; other geometries, however, may be used, e.g., where the target 308 matches the geometry of the gas chamber 330. In addition, as above, the reflective surface 302 may be a wall of the gas chamber 330, contained inside the gas chamber 330, or be disposed outside the gas chamber 330.

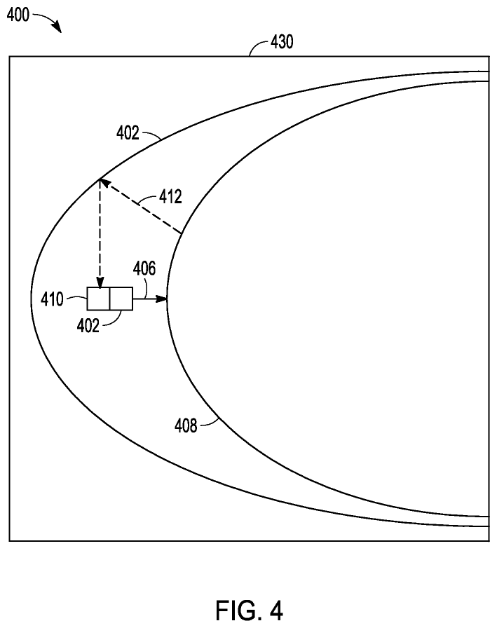

FIG. 4 shows a cross-sectional side view of an example of a gas sensing system 400, in accordance with some embodiments. As above, FIG. 4 omits the circuitry to operate electronics in the gas sensing system 400 in addition to the plumbing. The gas sensing system 400 contains a gas chamber 430 with a (smooth or textured) reflective surface 402, as well as a light emitter 404, light sensor 410, and a target 408. The target 408 may be prematurely truncated to form a truncated ellipsoid. In some embodiments, the truncation is a MWIR reflector. The target 408 can include a porous scattering material disposed therein, so that a gas sample can permeate hollow spaces within the porous scattering material of the target 408. As above, the light that enters the target 408 may scatter multiple times within the target 408 due to the porous scattering material. As in FIG. 3, the light emitter 404 and the light sensor 410 in FIG. 4 may be disposed adjacent to each other (e.g., back-to-back) at or near one of the foci of the ellipsoid. The light emitter 404 may emit light 406 generally in a direction toward the target 408, which propagates away from the light emitter 404, through the gas sample, in a range of propagation directions until the light 412 exits the target 408 and reaches the light sensor 410.

In the example of FIG. 4, the target 408 can match the geometry of the reflective surface 402; other geometries, however, may be used, e.g., where the target 408 matches the geometry of the gas chamber 430. In addition, as above, the reflective surface 402 may be a wall of the gas chamber 430, contained inside the gas chamber 430, or be disposed outside the gas chamber 430.

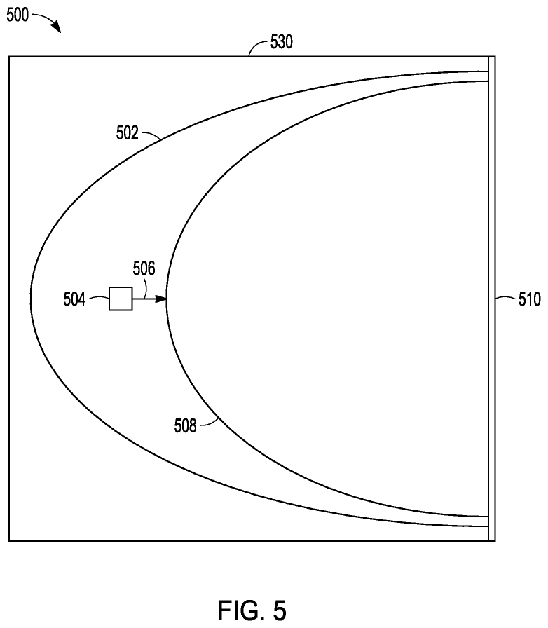

FIG. 5 shows a cross-sectional side view of an example of a gas sensing system 500, in accordance with some embodiments. As above, FIG. 5 omits the circuitry to operate electronics in the gas sensing system 500 in addition to the plumbing. The gas sensing system 500 contains a gas chamber 530 with a (smooth or textured) reflective surface 502, as well as a light emitter 504, light sensor 510, and a target 508. In FIG. 5, the reflective surface 502 and/or the target 508 may form a partial paraboloid, rather than a partial ellipsoid shown in FIG. 4. Specifically, the paraboloid has a central axis, such that a cross-section taken orthogonal to the central axis is generally circular, and a cross-section taken in a plane that includes the central axis is a parabola. The paraboloid includes one focus that is located along the central axis. The reflective surface 502 may be shaped such that every light ray or nearly every light ray that originates at the focus specularly reflects off the paraboloid version of the reflective surface 502 to be directed to be parallel or substantially parallel to the central axis. Further, the reflective surface 502 may be shaped such that each optical path from the focus to a plane that is orthogonal to the central axis, has a same or nearly a same value of optical path length.

The target 508 can include a porous scattering material disposed in therein, so that a gas sample can permeate hollow spaces within the porous scattering material of the target 508. The light emitter 504 can be a point-like emitter located at or near a focus of the paraboloid. The light 506 that enters the target 508 may scatter multiple times within the target 508 due to the porous scattering material. The light sensor 510 may be a large-area detector disposed at the open end of the paraboloid, as shown in FIG. 5. The light emitter 504 may emit light 506 generally in a direction toward the target 508, which propagates away from the light emitter 504, through the gas sample, in a range of propagation directions until the light 506 exits the target 508 and reaches the light sensor 510. The light sensor 510 may cover all, or only a portion, of the open portion of the paraboloid.

In the example of FIG. 5, the target 508 can match the geometry of the reflective surface 502; other geometries, however, may be used, e.g., where the target 508 matches the geometry of the gas chamber 530. In addition, as above, the reflective surface 502 may be a wall of the gas chamber 530, contained inside the gas chamber 530, or be disposed outside the gas chamber 530.

In the configuration of FIG. 5, the light emitter 504 is oriented to emit light generally toward the light sensor 510 (e.g., with an angular emission pattern that peaks along a central axis of the paraboloid and decreases at angles away from the central axis). However, a person of ordinary skill in the art will recognize that a light emitter that emits 360 degrees, or some subset thereof, may be used as well. A complete solid angle of 4 π steradians can also be considered. It is possible to use different orientations for the light emitter 504, which can illuminate different portions of the reflective surface 502, and in turn illuminate different portions of the volume of the gas chamber 530.

In an alternate arrangement, the light sensor 510 can be a point-like detector (i.e., small enough compared to the gas chamber 530 that the light sensor 510 is effectively a point) located at or near a focus of the paraboloid and the light emitter 504 may be a large-area emitter disposed at the open end of the paraboloid. In this case, the light emitter 504 may have an emitting area that is arranged generally orthogonal to the central axis of the paraboloid.

The configurations above can include a reflective surface that is generally smooth, so as to provide specular (e.g., non-diffusive) reflection. The reflective surface can be shaped as at least a portion of a quadric surface (e.g., a surface having a cross-section that is a conic section), such as an ellipsoid or a paraboloid, or may be arbitrarily shaped to all the gas detection systems to be provided in different sized and shaped devices. As above, all or part of reflective surface may have MWIR-rough (generating randomized reflection) or otherwise textured surface. Shaping the reflective surface in this manner can reduce or eliminate path-to-path variations in the lengths of the various optical paths that extend from the light emitter, through the gas sample and reflecting from the reflective surface, to the light sensor. By reducing or eliminating these optical path length variations, the system can more accurately use the Beer-Lambert Law to determine the concentration of the gas material in the gas sample.

In some embodiments, the cavity and/or the gas-permeable target may have any shape and the smooth or textured reflective surface may be shaped to form a volumetric reflector.

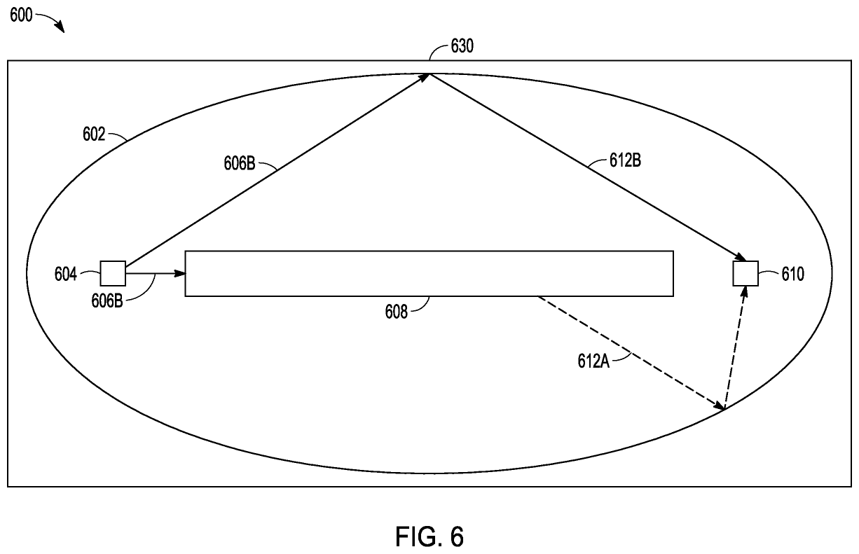

FIG. 6 shows a cross-sectional side view of an example of a gas sensing system 600, in accordance with some embodiments. As above, FIG. 6 omits the circuitry to operate electronics in the gas sensing system 600 in addition to the plumbing. The gas sensing system 600 contains a gas chamber 630 with a (smooth or textured) reflective surface 602, as well as a light emitter 604, light sensor 610, and a target 608. The target 608 can include a porous scattering material disposed in therein, so that a gas sample can permeate hollow spaces within the porous scattering material of the target 608. In this case, however, the target 608 may be disposed within only a relatively limited portion of the gas chamber 630. As shown, the target 608 may be disposed along the z axis, which is parallel to the direct optical path between the light emitter 604 and the light sensor 610. Thus, only this limited portion may be filled with scattering material, which may be either gas permeable or nonpermeable, and the remainder of the gas chamber 630 may be hollow. The target 608 may be cylindrical or any other shape disposed along the z axis between the light emitter 604 and the light sensor 610.

As above, the light 606a that enters the target 608 may scatter multiple times within the target 608 due to the porous scattering material before the scattered light 612a is received by the light sensor 610 (perhaps being reflected by the reflective surface 602). The light emitter 604 and the light sensor 610 in FIG. 6 may be disposed at or near different the foci of the ellipsoid. The light emitter 604 may also emit other light 606b generally in a direction toward the light emitter 604, but does not pass through the target 608. Instead, the other light 606b may pass through the gas in the remaining (hollow) area of the gas chamber 630, until reflected by the reflective surface 602 and the reflected light 612b reaches the light sensor 610.

Although the target 608 may be filled with scattering material, which may be either gas permeable or nonpermeable, and the remainder of the gas chamber 630 may be hollow, in some embodiments, the target 608 may be filled with non-gas permeable scattering material and the remainder of the gas chamber 630 may be filled with gas permeable scattering material.

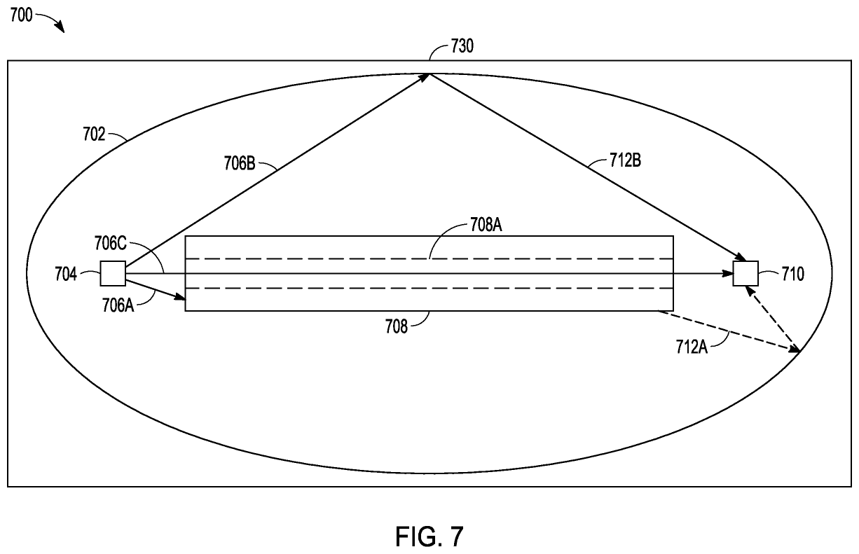

FIG. 7 shows a cross-sectional side view of an example of a gas sensing system 700, in accordance with some embodiments. The gas sensing system 700 of FIG. 7 is similar to that of FIG. 6. As above, FIG. 7 omits the circuitry to operate electronics in the gas sensing system 700 in addition to the plumbing. The gas sensing system 700 contains a gas chamber 730 with a (smooth or textured) reflective surface 702, as well as a light emitter 704, light sensor 710, and a target 708. The target 708 can include a porous scattering material disposed in therein, so that a gas sample can permeate hollow spaces within the porous scattering material of the target 708. In this case, however, while the target 708 may be disposed within only a relatively limited portion of the gas chamber 730, the center 708a, which provides a direct path between the light emitter 704 and the light sensor 710 may be hollow (i.e., not include porous scattering material). As shown, the target 708 may be disposed along the z axis, which is parallel to the direct optical path between the light emitter 704 and the light sensor 710. Thus, only this limited portion may be filled with scattering material, which may be either gas permeable or nonpermeable, and the remainder of the gas chamber 730 may be hollow. The target 708 may be cylindrical or any other shape disposed along the z axis between the light emitter 704 and the light sensor 710.

As above, the light 706a that enters the target 708 may scatter multiple times within the target 708 due to the porous scattering material before the scattered light 712a is received by the light sensor 710 (perhaps being reflected by the reflective surface 702). Direct light 706c that enters the center 708a may be received by the light sensor 710 without scattering. The light emitter 704 and the light sensor 710 in FIG. 7 may be disposed at or near different the foci of the ellipsoid. The light emitter 704 may also emit other light 706b generally in a direction toward the light emitter 704, but does not pass through the target 708. Instead, the other light 706b may pass through the gas in the remaining (hollow) area of the gas chamber 730, until reflected by the reflective surface 702 and the reflected light 712b reaches the light sensor 710.

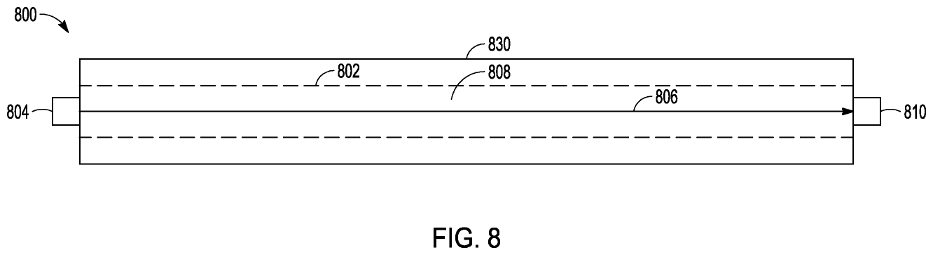

FIG. 8 shows a cross-sectional side view of an example of a gas sensing system 800, in accordance with some embodiments. The gas sensing system 800 of FIG. 8 is similar to that of FIG. 7. As above, FIG. 8 omits the circuitry to operate electronics in the gas sensing system 800 in addition to the plumbing. The gas sensing system 800 contains a gas chamber 830 with a (smooth or textured) reflective surface 802, as well as a light emitter 804, light sensor 810, and a target 808. The target 808 can include a porous scattering material disposed in therein, so that a gas sample can permeate hollow spaces within the porous scattering material of the target 808. As shown, the target 808 may be disposed along the z axis, which is parallel to the direct optical path between the light emitter 804 and the light sensor 810. Thus, only this limited portion of the gas chamber 830 may be filled with scattering material. The target 808 may be cylindrical or any other shape disposed along the z axis between the light emitter 804 and the light sensor 810. As above, the light 806 that enters the target 808 may scatter multiple times within the target 808 due to the porous scattering material before the scattered light is received by the light sensor 810.

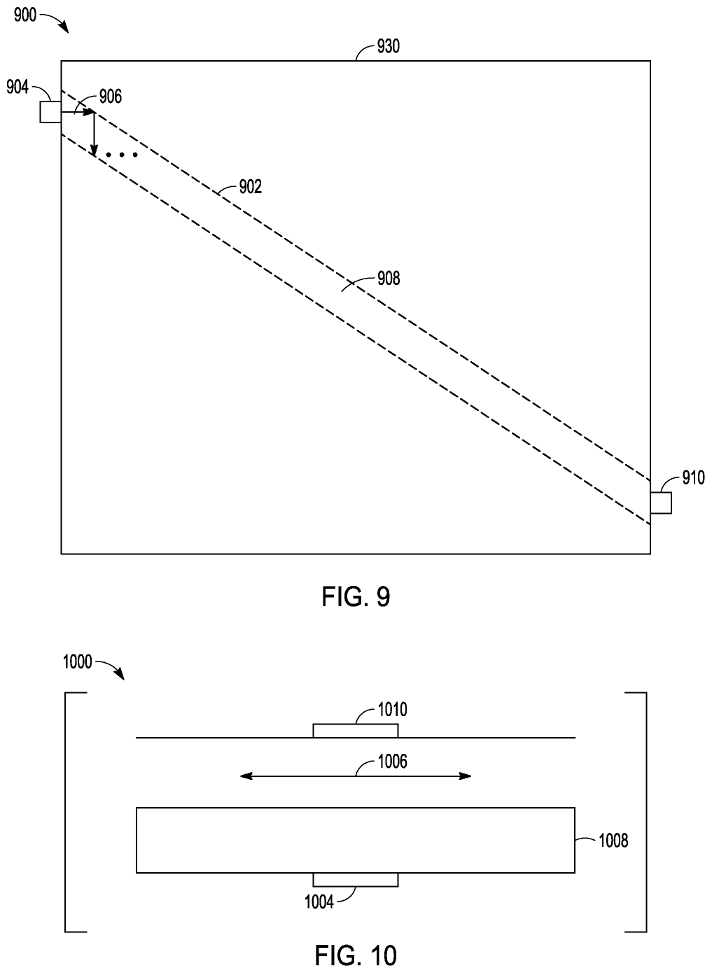

FIG. 9 shows a cross-sectional side view of an example of a gas sensing system 900, in accordance with some embodiments. The gas sensing system 900 of FIG. 9 is similar to that of FIG. 8, except the geometry of the cavity is different. As above, FIG. 9 omits the circuitry to operate electronics in the gas sensing system 900 in addition to the plumbing. The gas sensing system 900 contains a gas chamber 930 with a reflective surface 902, as well as a light emitter 904, light sensor 910, and a target 908. The target 908 can include a porous scattering material disposed in therein, so that a gas sample can permeate hollow spaces within the porous scattering material of the target 908. Thus, only this limited portion of the gas chamber 930 may be filled with scattering material, or the target 908 may be hollow. The target 908 may be cylindrical or any other shape disposed between the light emitter 904 and the light sensor 910. As above, the light 906 that enters the target 908 may scatter multiple times within the target 908 due to the porous scattering material before the scattered light is received by the light sensor 910. The reflective surface 902 may be smooth or may be provide diffusive scattering.

FIG. 10 shows a cross-sectional side view of an example of a gas sensing system 1000, in accordance with some embodiments. The gas sensing system 1000 contains a light emitter 1004, light sensor 1010, and a scattering medium 1008. The gas sample 1006 is introduced in a hollow region of the gas chamber adjacent to the light sensor 1010. The scattering medium 1008 fills only a portion of the cavity adjacent to the light emitter 1004. As above, the light that enters the scattering medium 1008 may scatter multiple times within the scattering medium 1008 before the scattered light is received by the light sensor 1010. The light sensor 1010 may be limited to about the same size as the light emitter 1004 or may be a wide area sensor that extends along most or all of the (as shown) upper wall of the gas chamber. The scattering medium 1008 may be porous or non-porous, gas permeable or non-gas permeable, as desired.

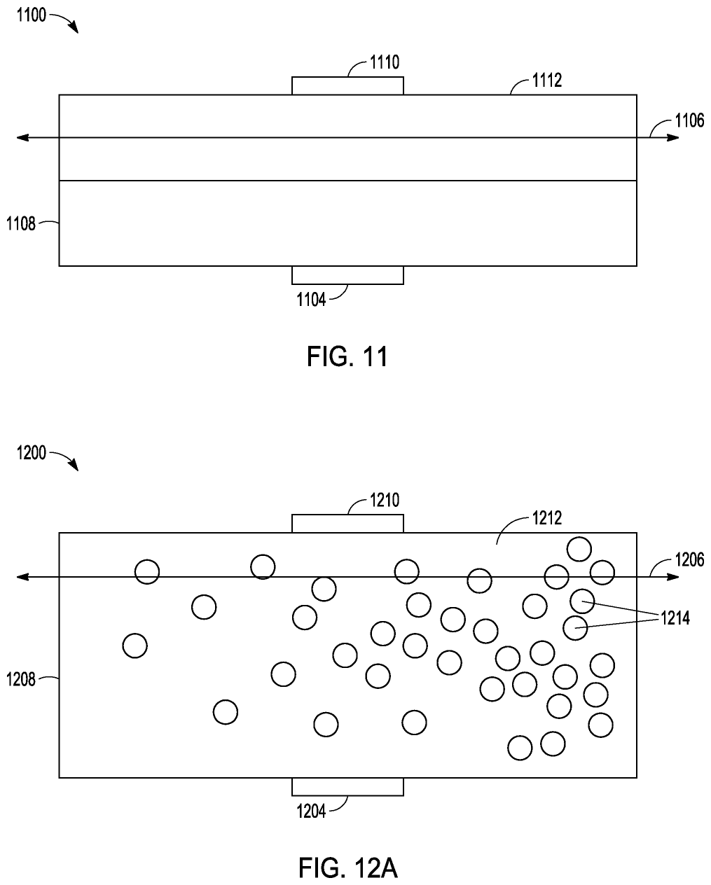

FIG. 11 shows a cross-sectional side view of an example of a gas sensing system 1100, in accordance with some embodiments. The gas sensing system 1100 contains a light emitter 1104, light sensor 1110, a porous scattering medium 1112 and a non-porous scattering medium 1108. The gas sample 1106 is introduced into the porous scattering medium 1112, which is adjacent to the light sensor 1110. The non-porous scattering medium 1108 fills only a portion of the cavity adjacent to the light emitter 1104, with the remaining portion filled by the porous scattering medium 1112. In other embodiments, a hollow region may also be introduced into the gas chamber of FIG. 11. As above, the light that enters the non-porous scattering medium 1108 may scatter multiple times within the non-porous scattering medium 1108 before the scattered light is received by the light sensor 1110. The light sensor 1110 may be limited to about the same size as the light emitter 1104 or may be a wide area sensor that extends along most or all of the (as shown) upper wall of the gas chamber. The non-porous scattering medium 1108 may be porous or non-porous, gas permeable or non-gas permeable, as desired.

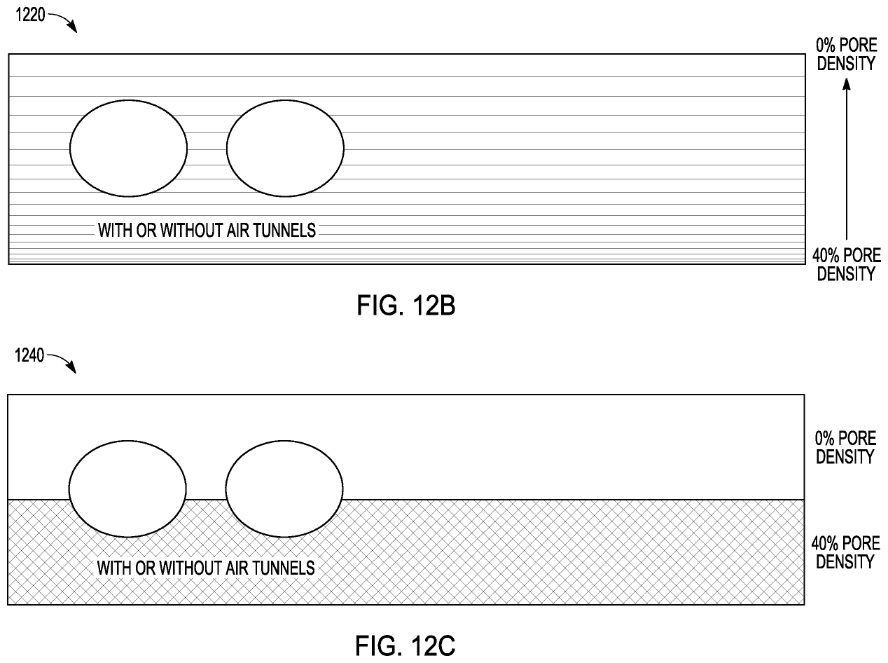

FIG. 12A shows a cross-sectional side view of an example of a gas sensing system 1200, in accordance with some embodiments. The gas sensing system 1200 contains a light emitter 1204, light sensor 1210, and a gas-permeable porous scattering medium 1208. The gas sample 1206 is introduced into the porous scattering medium 1208, which fills the cavity between the light sensor 1210 and the light emitter 1204. In other embodiments, a hollow region may also be introduced into the gas chamber of FIG. 12A. As above, the light that enters the porous scattering medium 1208 may scatter multiple times within the porous scattering medium 1208 before the scattered light is received by the light sensor 1210. The light sensor 1210 may be limited to about the same size as the light emitter 1204 or may be a wide area sensor that extends along most or all of the (as shown) upper wall of the gas chamber.

The porous scattering medium 1208 may have a base material 1212 that is transparent to the light emitted by the light emitter 1204 and hollow areas (or areas filled with a material of a different refractive index as the base material) 1214. The porous scattering medium 1208 may have a porosity gradient (e.g., zero porosity at in one section and non-zero porosity in another). This gradient may be any of one or more of linear (as shown in the gas sensing system 1220 of FIG. 12B), exponential, or discontinuous (step, as shown in the gas sensing system 1240 of FIG. 12C), for example. Although FIG. 12A shows the hollow areas 1214 are formed from spheres, the hollow areas 1214 may have any shape. In addition, multiple gradients may be present within the porous scattering medium 1208. The pore gradient may change from 0% to about 40% by volume, although this range is not so limited and may start and/or stop at different % by volume. In addition, as shown in FIGS. 12B and 12C, the porous scattering medium 1208 may or may not have hollow tubes (air tunnels) that extend through the porous scattering medium 1208.

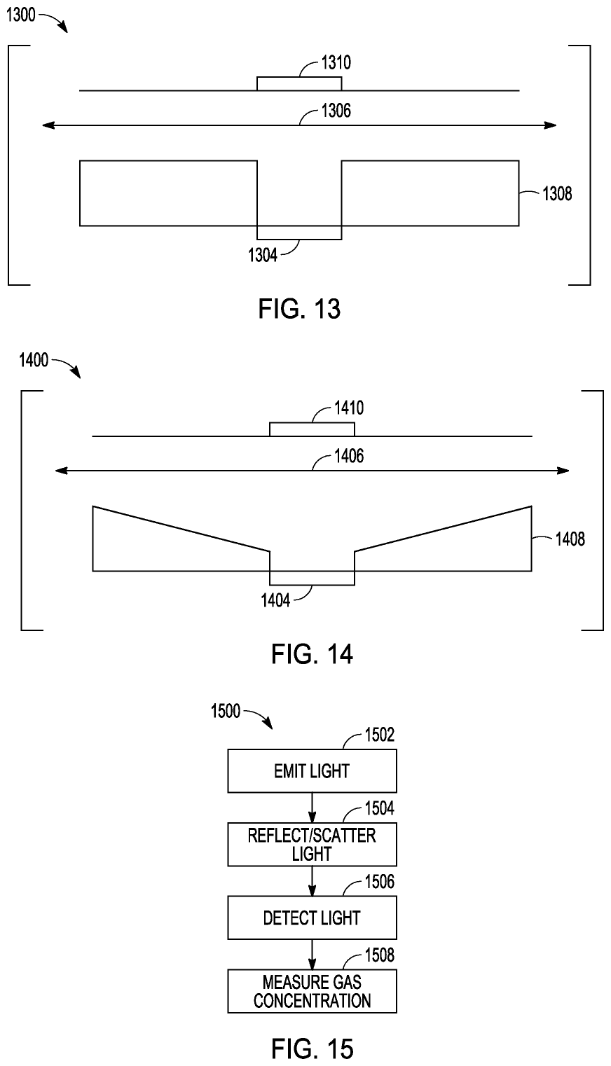

FIG. 13 shows a cross-sectional side view of an example of a gas sensing system 1300, in accordance with some embodiments. The gas sensing system 1300 contains a light emitter 1304, light sensor 1310, and a discontinuous scattering medium 1308. The gas sample 1306 is introduced in a hollow region of the gas chamber adjacent to the light sensor 1310. The discontinuous scattering medium 1308 fills only a portion of the cavity adjacent to the light emitter 1304. As above, the light that enters the discontinuous scattering medium 1308 may scatter multiple times within the discontinuous scattering medium 1308 before the scattered light is received by the light sensor 1310. The light sensor 1310 may be limited to about the same size as the light emitter 1304 or may be a wide area sensor that extends along most or all of the (as shown) upper wall of the gas chamber. The discontinuous scattering medium 1308 may be porous or non-porous, gas permeable or non-gas permeable, as desired. The discontinuous scattering medium 1308 may be porous or non-porous, gas permeable or non-gas permeable, as desired. The discontinuous scattering medium 1308 may have a substantially uniform cross-section in a width direction (from the light emitter 1304 to the light sensor 1310) but is discontinuous such that an area directly between the light emitter 1304 and the light sensor 1310 does not contain the discontinuous scattering medium 1308.

FIG. 14 shows a cross-sectional side view of an example of a gas sensing system 1400, in accordance with some embodiments. The gas sensing system 1400 contains a light emitter 1404, light sensor 1410, and a discontinuous scattering medium 1408. The gas sample 1406 is introduced in a hollow region of the gas chamber adjacent to the light sensor 1410. The discontinuous scattering medium 1408 fills only a portion of the cavity adjacent to the light emitter 1404. As above, the light that enters the discontinuous scattering medium 1408 may scatter multiple times within the discontinuous scattering medium 1408 before the scattered light is received by the light sensor 1410. The light sensor 1410 may be limited to about the same size as the light emitter 1404 or may be a wide area sensor that extends along most or all of the (as shown) upper wall of the gas chamber. The discontinuous scattering medium 1408 may be porous or non-porous, gas permeable or non-gas permeable, as desired. The discontinuous scattering medium 1408 may be porous or non-porous, gas permeable or non-gas permeable, as desired. The discontinuous scattering medium 1408 may have cross-section that tapers in the width direction and is discontinuous such that the area directly between the light emitter 1404 and the light sensor 1410 does not contain the discontinuous scattering medium 1408. The taper of the discontinuous scattering medium 1408 may be linear and stair-step and may extend from opposing ends of the discontinuous scattering medium 1408 or may extend from an intermediate point between the end of the discontinuous scattering medium 1408 and the nearest edge of the light emitter 1404.

The flow direction of a gas of interest is not limited by these topologies; that is, the gas inlet and outlet locations (such as that shown in FIG. 2) with respect to emitter and sensor are not limited by these topologies. In some embodiments, the porous material may be enhanced to increase adsorption of target gas molecules and thereby artificially increase the concentration of target gas inside the scattering medium (and thus increase the amount of IR absorbed, thereby improving effective signal).

In the above embodiments, the gas-permeable material may include porous ceramic, micropipes (having a diameter of about, for example, 1-10 microns) made of MWIR-transparent or translucent material (e.g., sapphire), or empty tunnels or openings that are contiguous or separate from an ambient environment external to the porous scattering material to allow faster distribution of gas throughout the sensing medium (cavity).

FIG. 15 shows a flow chart of an example of a method 1500 for measuring a concentration of a gas in a gas sample, in accordance with some embodiments. The gas can have an absorption peak at a first wavelength. The method can be executed on any of the gas sensing systems discussed herein, or on other suitable gas sensing systems. The flowchart of the method 1500 is merely exemplary, additional operations may be present, but are not shown for convenience.

At operation 1502, the method 1500 may include emitting, using a light emitter, light having a spectrum that includes the first wavelength.

At operation 1504, the method 1500 can include reflecting and/or scattering at least some of the light from multiple surfaces of a porous scattering material within, as well as a reflective surface of, the gas chamber. The porous scattering material contains the gas to be detected. The reflective surface is concave and has a shape that is at least a portion of a quadric surface.

At operation 1506, the method 1500 can include detecting, a detector that detects the light that has passed through the portion of the gas chamber (multiple times) and not been absorbed by the gas, at least some of the light that passed through the porous scattering material.

At operation 1508, the concentration of the gas is measured determined by at least one processor using the techniques described above. The system, for example, may be calibrated for each gas of interest using known gas concentrations before being installed in the commercial apparatus or afterwards but before being sold commercially to a consumer. The processor may be connected to a storage device that includes a non-transitory machine readable medium (hereinafter simply referred to as machine readable medium) on which is stored one or more sets of data structures or instructions (e.g., software) embodying or utilized by any one or more of the techniques or functions described herein. While the machine readable medium may be a single medium, the term “machine readable medium” may include a single medium or multiple media (e.g., a centralized or distributed database, and/or associated caches and servers) configured to store the one or more instructions.

The term “machine readable medium” may include any medium that is capable of storing, encoding, or carrying instructions for execution by the processor that cause the processor to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. The instructions may further be transmitted or received over a communications network using a transmission medium via a network interface device utilizing any one of a number of wireless local area network (WLAN) transfer protocols. The term “processor circuitry” or “processor” as used herein thus refers to, is part of, or includes circuitry capable of sequentially and automatically carrying out a sequence of arithmetic or logical operations, or recording, storing, and/or transferring digital data. The term “processor circuitry” or “processor” may refer to one or more application processors, one or more baseband processors, a physical central processing unit (CPU), a single- or multi-core processor, and/or any other device capable of executing or otherwise operating computer-executable instructions, such as program code, software modules, and/or functional processes.

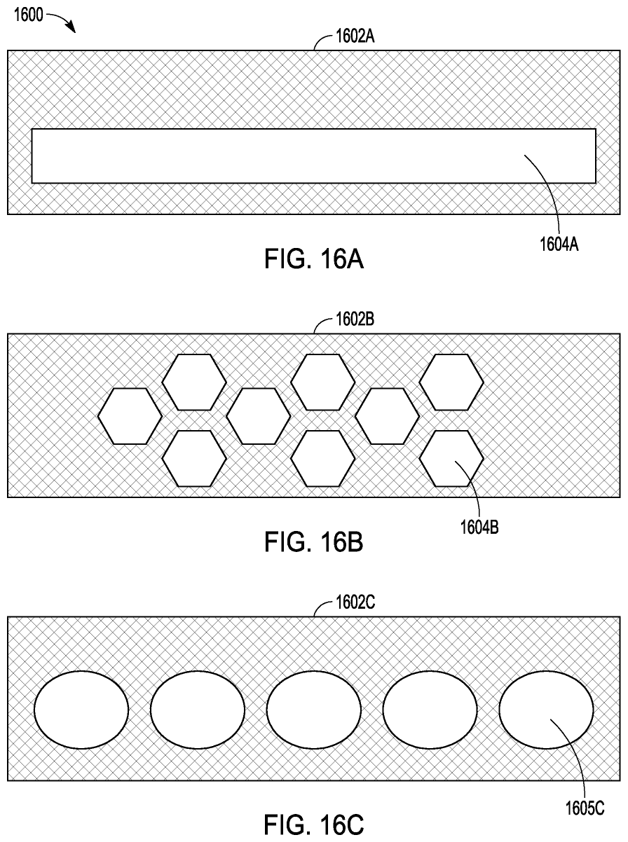

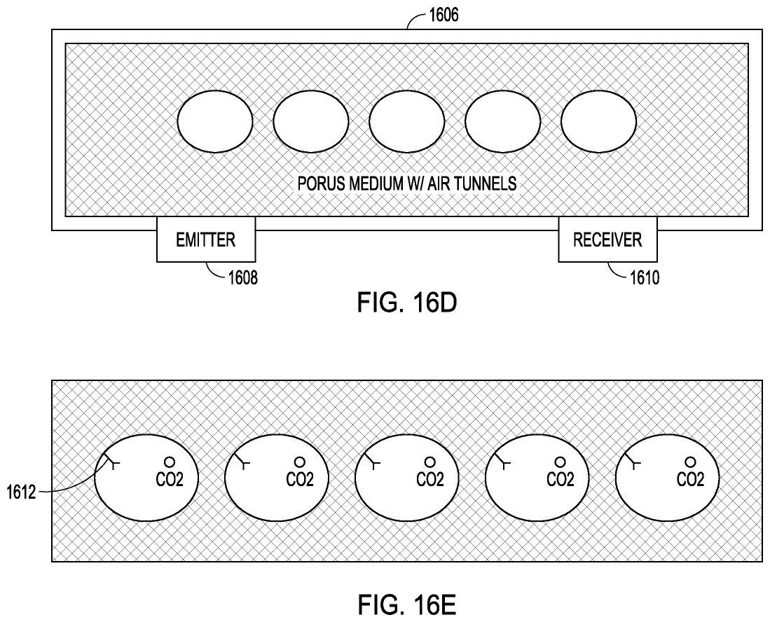

FIGS. 16A-16E show cross-sectional side views of examples of a gas sensing system 1600, in accordance with some embodiments. Each of the gas chambers may have a porous structure 1602a, 1602b, 1602c that surrounds a gas flow structure 1604a, 1604b, 1604c. The gas flow structure 1604a, 1604b, 1604c may have different shapes, such as those shown in FIGS. 16A-16C: a single large cavity (gas flow structure 1604a), a honeycomb structure (gas flow structure 1604b), or a cylindrical channel structure (gas flow structure 1604c). The gas flow structure 1604a, 1604b, 1604c may be hollow or may contain a gas-permeable material. The porous structure 1602a, 1602b, 1602c may have smooth or textured (e.g., diffusive) reflective layers 1606 (as shown in FIG. 16D) and may contain a gas-permeable material. The reflective layers 1606 may fully encase or be disposed on a combination of the sides of the chamber to trap and recycle light. The reflective layers 1606 may have openings for the light emitter 1608 and light receiver (sensor) 1610. In some cases, as shown in FIG. 16E, the inner surface of the gas flow structure 1604a, 1604b, 1604c may be treated to aid in gas detection. That is, the interior pore surface may be treated with a gas-binding material 1612 to increase gas concentration around the light path. Examples of the gas-binding material 1612 may include zeolite-like materials coated onto the surface as a nanocrystalline slurry or grown in a layer-by-layer synthesis. The gas-binding material 1612 may also be infused into a porous material.

### EXAMPLES

Example 1 is a gas sensing system configured to measure a concentration of a gas introduced therein, the gas having an absorption peak at a first wavelength, the gas sensing system comprising: an emitter configured to emit light having a spectrum that includes, at least the first wavelength; a sensor configured to detect at least some of the light emitted by the emitter; a gas chamber including a reflective surface having a concave portion, the reflective surface configured to redirect the light from the emitter towards the sensor; and a porous scattering material disposed within the gas chamber, the porous scattering material being substantially transparent at the first wavelength, the porous scattering material configured to scatter at least some of the light.

In Example 2, the subject matter of Example 1 includes, wherein: the reflective surface is an ellipsoid; the ellipsoid includes a first focus and a second focus; the emitter is proximate to the first focus; and the sensor is proximate to the second focus.

In Example 3, the subject matter of any one of Examples 1-2 includes, wherein: the reflective surface is an ellipsoid; the ellipsoid includes a first focus and a second focus; the emitter is proximate to the first focus; and the sensor is proximate to the first focus.

In Example 4, the subject matter of Examples 1-3 includes, wherein: the reflective surface is a paraboloid; the paraboloid includes a focus; the emitter is proximate to the focus; and the sensor extends substantially across an open end of the paraboloid.

In Example 5, the subject matter of Examples 1-4 includes, wherein: the reflective surface is at least a partial paraboloid; the at least the partial paraboloid includes a focus; the sensor is proximate to the focus; and the emitter extends substantially across an open end of the at least the partial paraboloid.

In Example 6, the subject matter of Examples 1-5 includes, wherein: the reflective surface is a paraboloid; the paraboloid includes a central axis and a focus located on the central axis; the sensor is located proximate the focus; and the emitter has an emitting area that is arranged generally orthogonal to the central axis.

In Example 7, the subject matter of Examples 1-6 includes, wherein at least some of the reflective surface is roughened to randomize reflection of light of Middle wavelength Infrared (MWIR) wavelengths.

In Example 8, the subject matter of Examples 1-7 includes, wherein: a cavity within the gas chamber contains the emitter and the sensor; the porous scattering material forms the cavity; and the porous scattering material is permeable to the gas.

In Example 9, the subject matter of Examples 1-8 includes, wherein: a cavity within the gas chamber contains the emitter and the sensor; the porous scattering material forms the cavity; and the porous scattering material is not permeable to the gas.

In Example 10, the subject matter of Examples 1-9 includes, wherein: a cavity within the gas chamber contains the emitter and the sensor; the porous scattering material is disposed along a direct optical path between the emitter and the sensor; and a portion of the cavity that does not contain the porous scattering material is hollow.

In Example 11, the subject matter of Examples 1-10 includes, wherein: a cavity within the gas chamber contains the emitter and the sensor; the porous scattering material is disposed in a first portion of the cavity that extends in a direction of a direct optical path between the emitter and the sensor; a direct optical path between the emitter and the sensor is hollow; and a second portion of the cavity that does not contain the porous scattering material is hollow.

In Example 12, the subject matter of Examples 1-11 includes, wherein: a cavity within the gas chamber contains the emitter and the sensor; and the porous scattering material comprises: first porous scattering material disposed in a first portion of the cavity that extends in a direction of a direct optical path between the emitter and the sensor, the first porous scattering material is not permeable to the gas, and second porous scattering material disposed in a second portion of the cavity, the second porous scattering material is permeable to the gas.

In Example 13, the subject matter of Examples 1-12 includes, wherein the porous scattering material is permeable to the gas and includes at least one selected from a group that includes: porous ceramic, micropipes comprising Middle wavelength Infrared (MWIR)-transparent or translucent material, and hollow tunnels or openings that are contiguous or separate from an ambient environment external to the porous scattering material.

In Example 14, the subject matter of Examples 1-13 includes, wherein the porous scattering material is permeable to the gas and has a porosity gradient.

In Example 15, the subject matter of Examples 1-14 includes, at least one processor configured to determine a concentration of the gas from an intensity of light that impinges the sensor.

Example 16 is a gas sensing system configured to measure a concentration of a gas introduced therein, the gas having an absorption peak at a first wavelength, the gas sensing system comprising: an emitter configured to emit light having a spectrum that includes, at least the first wavelength; a sensor configured to detect at least some of the light emitted by the emitter; a gas chamber including a reflective surface having a concave portion, the reflective surface configured to redirect the light from the emitter towards the sensor; a porous scattering material disposed within the gas chamber, the porous scattering material being substantially transparent at the first wavelength, the porous scattering material configured to scatter at least some of the light; and at least one processor configured to determine a concentration of the gas from an intensity of light that impinges the sensor from a Beer-Lambert Law.

In Example 17, the subject matter of Example 16 includes, wherein the porous scattering material is permeable to the gas and has a porosity gradient.

Example 18 is a method for measuring a concentration of a gas introduced into a gas chamber, the gas having an absorption peak at a first wavelength, the method comprising: emitting first light having a spectrum that includes, the first wavelength; redirecting, from a reflective surface of the gas chamber, at least some of the first light to form reflected light, the reflective surface being concave; scattering, at least some of the first light, by a porous scattering material that is disposed within the gas chamber, the porous scattering material being substantially transparent at the first wavelength to form scattered light; and detecting, at a sensor, at least some of the scattered light and reflected light.

In Example 19, the subject matter of Example 18 includes, determining a concentration of the gas, using at least one processor, from an intensity of light that impinges the sensor from a Beer-Lambert Law.

In Example 20, the subject matter of Examples 18-19 includes, wherein the porous scattering material is permeable to the gas.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

As above, elements in the drawings are not necessarily drawn to scale. The configurations shown in the drawings are merely examples and should not be construed as limiting the scope of the disclosed subject matter in any manner.

While exemplary embodiments of the present disclosed subject matter have been shown and described herein, it will be obvious to those skilled in the art that such embodiments are provided by way of example only. Numerous variations, changes, and substitutions will now occur to those skilled in the art, upon reading and understanding the material provided herein, without departing from the disclosed subject matter. It should be understood that various alternatives to the embodiments of the disclosed subject matter described herein may be employed in practicing the various embodiments of the subject matter. It is intended that the following claims define the scope of the disclosed subject matter and that methods and structures within the scope of these claims and their equivalents be covered thereby.

What is claimed is:

1. A gas sensing system configured to measure a concentration of a gas introduced therein, the gas having an absorption peak at a first wavelength, the gas sensing system comprising:
an emitter configured to emit light having a spectrum that includes at least the first wavelength;
a sensor configured to detect at least some of the light emitted by the emitter;
a gas chamber including a reflective surface having a concave portion, the reflective surface configured to redirect the light from the emitter towards the sensor; and
a porous scattering material disposed within the gas chamber, the porous scattering material being substantially transparent at the first wavelength, the porous scattering material configured to scatter at least some of the light.

2. The gas sensing system of claim 1, wherein:
the reflective surface is an ellipsoid;
the ellipsoid includes a first focus and a second focus;
the emitter is proximate to the first focus; and
the sensor is proximate to the second focus.

3. The gas sensing system of claim 1, wherein:
the reflective surface is an ellipsoid;
the ellipsoid includes a first focus and a second focus;
the emitter is proximate to the first focus; and
the sensor is proximate to the first focus.

4. The gas sensing system of claim 1, wherein:
the reflective surface is a paraboloid;
the paraboloid includes a focus;
the emitter is proximate to the focus; and
the sensor extends substantially across an open end of the paraboloid.

5. The gas sensing system of claim 1, wherein:
the reflective surface is at least a partial paraboloid;
the at least the partial paraboloid includes a focus;
the sensor is proximate to the focus; and
the emitter extends substantially across an open end of the at least the partial paraboloid.

6. The gas sensing system of claim 1, wherein:
the reflective surface is a paraboloid;
the paraboloid includes a central axis and a focus located on the central axis;
the sensor is located proximate the focus; and
the emitter has an emitting area that is arranged generally orthogonal to the central axis.

7. The gas sensing system of claim 1, wherein at least some of the reflective surface is roughened to randomize reflection of light of Middle wavelength Infrared (MWIR) wavelengths.

8. The gas sensing system of claim 1, wherein:
a cavity within the gas chamber contains the emitter and the sensor;
the porous scattering material forms the cavity; and
the porous scattering material is permeable to the gas.

9. The gas sensing system of claim 1, wherein:
a cavity within the gas chamber contains the emitter and the sensor;
the porous scattering material forms the cavity; and
the porous scattering material is not permeable to the gas.

10. The gas sensing system of claim 1, wherein:
a cavity within the gas chamber contains the emitter and the sensor;
the porous scattering material is disposed along a direct optical path between the emitter and the sensor; and
a portion of the cavity that does not contain the porous scattering material is hollow.

11. The gas sensing system of claim 1, wherein:
a cavity within the gas chamber contains the emitter and the sensor;
the porous scattering material is disposed in a first portion of the cavity that extends in a direction of a direct optical path between the emitter and the sensor;
a direct optical path between the emitter and the sensor is hollow; and
a second portion of the cavity that does not contain the porous scattering material is hollow.

12. The gas sensing system of claim 1, wherein:
a cavity within the gas chamber contains the emitter and the sensor; and
the porous scattering material comprises:
first porous scattering material disposed in a first portion of the cavity that extends in a direction of a direct optical path between the emitter and the sensor, the first porous scattering material is not permeable to the gas, and
second porous scattering material disposed in a second portion of the cavity, the second porous scattering material is permeable to the gas.

13. The gas sensing system of claim 1, wherein the porous scattering material is permeable to the gas and includes at least one selected from a group that includes: porous ceramic, micropipes comprising Middle wavelength Infrared (MWIR)-transparent or translucent material, and hollow tunnels or openings that are contiguous or separate from an ambient environment external to the porous scattering material.

14. The gas sensing system of claim 1, wherein the porous scattering material is permeable to the gas and has a porosity gradient.

15. The gas sensing system of claim 1, further comprising at least one processor configured to determine a concentration of the gas from an intensity of light that impinges the sensor.

16. A gas sensing system configured to measure a concentration of a gas introduced therein, the gas having an absorption peak at a first wavelength, the gas sensing system comprising:
an emitter configured to emit light having a spectrum that includes at least the first wavelength;
a sensor configured to detect at least some of the light emitted by the emitter;
a gas chamber including a reflective surface having a concave portion, the reflective surface configured to redirect the light from the emitter towards the sensor;
a porous scattering material disposed within the gas chamber, the porous scattering material being substantially transparent at the first wavelength, the porous scattering material configured to scatter at least some of the light; and
at least one processor configured to determine a concentration of the gas from an intensity of light that impinges the sensor from a Beer-Lambert Law.

17. The gas sensing system of claim 16, wherein the porous scattering material is permeable to the gas and has a porosity gradient.

18. A method for measuring a concentration of a gas introduced into a gas chamber, the gas having an absorption peak at a first wavelength, the method comprising:
emitting first light having a spectrum that includes the first wavelength;
redirecting, from a reflective surface of the gas chamber, at least some of the first light to form reflected light, the reflective surface being concave;
scattering, at least some of the first light, by a porous scattering material that is disposed within the gas chamber, the porous scattering material being substantially transparent at the first wavelength to form scattered light; and
detecting, at a sensor, at least some of the scattered light and reflected light.

19. The method of claim 18, further comprising determining a concentration of the gas, using at least one processor, from an intensity of light that impinges the sensor from a Beer-Lambert Law.

20. The method of claim 18, wherein the porous scattering material is permeable to the gas.
